(19) United States
(12) Patent Application Publication
(10) Pub. No.: US 2020/0138615 A1
(43) Pub. Date: May 7, 2020

(54) Shoulder Abduction Device

(71) Applicant: Guy Kennedy, Arvada, CO (US)

(72) Inventor: Guy Kennedy, Arvada, CO (US)

(21) Appl. No.: 16/177,625

(22) Filed: Nov. 1, 2018

(51) Int. Cl. A61F 5/01

(52) U.S. Cl. CPC A61F 5/0193

(57) ABSTRACT

A shoulder abduction device having an integral storage compartment includes a block that is padded. A cavity extends into the block from an upper face of the block and is configured to stow an article. Each of opposing endpoints of a strap is selectively couplable to a respective opposing end of the block. The strap is configured to loopedly position around a waist of a user, positioning the opposing endpoints to couple to the block so that the block and the strap are coupled to the user with the block positioned on a side of a torso of the user. The block is configured to abduct a shoulder of the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relate to abduction devices and more particularly pertains to a new abduction device having an integral storage compartment.

### BRIEF SUMMARY OF THE INVENTION

An embodiment of the disclosure meets the needs presented above by generally comprising a block that is padded. A cavity extends into the block from an upper face of the block and is configured to stow an article. Each of opposing endpoints of a strap is selectively couplable to a respective opposing end of the block. The strap is configured to loopedly position around a waist of a user, positioning the opposing endpoints to couple to the block so that the block and the strap are coupled to the user with the block positioned on a side of a torso of the user. The block is configured to abduct a shoulder of the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a shoulder abduction device according to an embodiment of the disclosure.

FIG. 2 is a top view of an embodiment of the disclosure.

FIG. 3 is a side view of an embodiment of the disclosure.

FIG. 4 is a top view of an embodiment of the disclosure.

FIG. 5 is an in-use view of an embodiment of the disclosure.

### DETAILED DESCRIPTION OF THE INVENTION

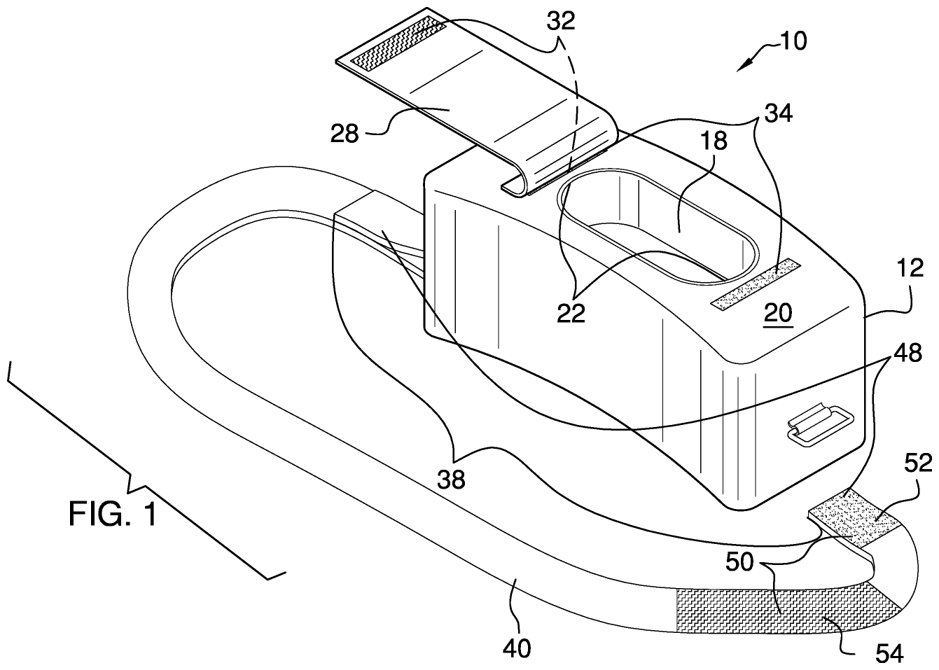

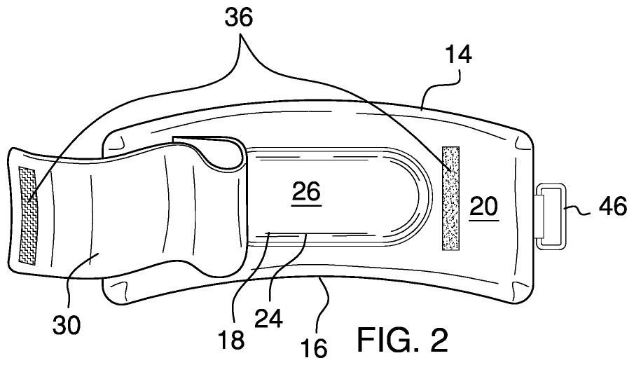

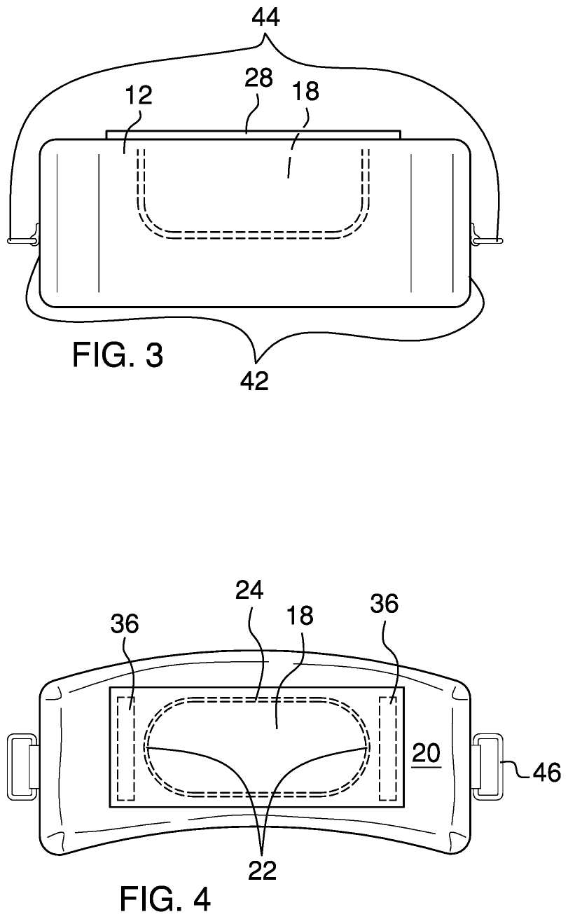

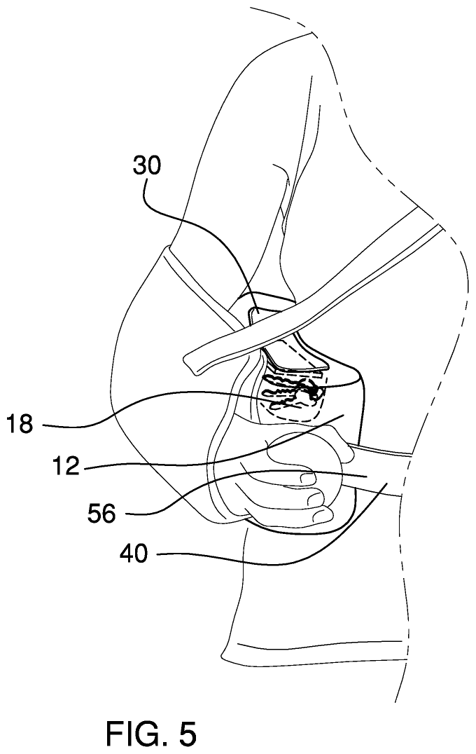

With reference now to the drawings, and in particular to FIGS. 1 through 5 thereof, a new abduction device embodying the principles and concepts of an embodiment of the disclosure and generally designated by the reference numeral 10 will be described.

As best illustrated in FIGS. 1 through 5, the shoulder abduction device 10 generally comprises a block 12 that is padded. The block 12 comprises foamed elastomer, or the like, so that the block 12 is resiliently compressible. The block 12 is substantially rectangularly box shaped. The block 12 has a first face 14, which is convexly arcuate, and a second face 16, which is concavely arcuate. The second face 16 is configured to abut a side of a torso of a user. The block 12 being resiliently compressible increases comfort of the user.

A cavity 18 extends into the block 12 from an upper face 20 of the block 12 and is configured to stow an article, such as a phone, keys, or the like. The cavity 18 has opposing termini 22 that are arcuate. The rounding of the termini 22 of the cavity 18 facilitates the user in locating and grasping the article that is positioned therein.

A liner 24 is coupled to an interior surface 26 of the block 12 that is defined by the cavity 18. The liner 24 comprises rubber. The liner 24 is configured to dampen noise that is produced by movement of the article in the cavity 18 and to protect the article from damage.

A lid 28 is selectively couplable to the upper face 20 of the block 12 to close the cavity 18. The lid 28 comprises a panel 30 that is flexible. A plurality of first connectors 32 is coupled to the panel 30. A plurality of second connectors 34 is coupled to the upper face 20 of the block 12. The second connectors 34 are complementary to the first connectors 32. Each second connector 34 is positioned to selectively couple to a respective first connector 32 to couple the panel 30 to the block 12 to close the cavity 18.

The plurality of second connectors 34 comprises two second connectors 34 that are positioned singly proximate to the opposing termini 22 of the cavity 18. The positioning of the two second connectors 34 allows the device 10 to be positioned on the left side or the right side the torso of the user with the cavity 18 being equally accessible to the user. The second connector 34 and the respective first connector 32 comprise a cover hook and loop fastener 36. The cover hook and loop fastener 36 is readily openable and closable by the user using a single hand.

Each of opposing endpoints 38 of a strap 40 is selectively couplable to a respective opposing end 42 of the block 12. The strap 40 is configured to loopedly position around a waist of the user, positioning the opposing endpoints 38 to couple to the block 12 so that the block 12 and the strap 40 are coupled to the user with the block 12 positioned on the side of the torso of the user. The block 12 is configured to abduct a shoulder of the user.

Each of a pair of first couplers 44 is coupled to a respective opposing end 42 of the block 12. Each first coupler 44 comprises a ring 46 that is hingedly coupled to the respective opposing end 42 of the block 12. The ring 46 is squarely shaped.

Each of a pair of second couplers 48 is coupled to a respective opposing endpoint 38 of the strap 40. The second couplers 48 are complementary to the first couplers 44. Each second coupler 48 is positioned to selectively couple to a respective first coupler 44 to couple the strap 40 and the block 12 around the waist of the user so that the block 12 is positioned on the side of the torso of the user. The block 12 is configured to abduct the shoulder of the user and the cavity 18 is configured to stow the article.

Each second coupler 48 comprises a strapping hook and loop fastener 50. A first element 52 of the strapping hook and loop fastener 50 is positioned adjacent to the respective opposing endpoint 38 of the strap 40. A second element 54 of the strapping hook and loop fastener 50 is positioned on the strap 40 distal from the first element 52. The strap 40 is positioned to insert through the ring 46, positioning the first element 52 to selectively couple to the second element 54 to removably couple the strap 40 to the ring 46.

The second element 54 is dimensionally longer than the first element 52 so that the first element 52 is selectively positionable on the second element 54 to adjustably couple the strap 40 and the block 12 around the waist of the user. The strapping hook and loop fasteners 50 are used to adjust an effective length 56 of the strap 40 so that the strap 40 and the block 12 fit comfortably around the waist of the user.

In use, one opposing endpoint 38 of the strap 40 is coupled to the block 12. The block 12 is positioned on the side of the torso of the user and the strap 40 is loopedly positioned around the waist of the user, positioning the other opposing endpoint 38 of the strap 40 to couple to the block 12. The block 12 is configured to abduct the shoulder of the user. The lid 28 is positioned to removably couple to the upper face 20 of the block 12, allowing the user to stow and retrieve the article from the cavity 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A shoulder abduction device comprising:
a block, the block being padded;
a cavity extending into the block from an upper face of the block wherein the cavity is configured for stowing an article; and
a strap having opposing endpoints, each opposing endpoint being selectively couplable to a respective opposing end of the block wherein the strap is configured for loopedly positioning around a waist of a user positioning the opposing endpoints for coupling to the block such that the block and the strap are coupled to the user and such that the block is positioned on a side of a torso of the user wherein the block is configured for abducting a shoulder of the user.

2. The device of claim 1, further including the block comprising foamed elastomer such that the block is resiliently compressible.

3. The device of claim 1, further including the block being substantially rectangularly box shaped, the block having a first face and a second face, the first face being convexly arcuate, the second face being concavely arcuate wherein the second face is configured abutting the side of the torso of the user.

4. The device of claim 1, further including the cavity having opposing termini, the opposing termini being arcuate.

5. The device of claim 1, further including a liner coupled to an interior surface of the block defined by the cavity, the liner comprising rubber wherein the liner is configured for dampening noise produced by movement of the article in the cavity and for protecting the article from damage.

6. The device of claim 1, further including a lid selectively couplable to the upper face of the block for closing the cavity.

7. The device of claim 6, further including the lid comprising:
a panel, the panel being flexible;
a plurality of first connectors coupled to the panel; and
a plurality of second connectors coupled to the upper face of the block, the second connectors being complementary to the first connectors wherein each second connector is positioned for selectively coupling to a respective first connector for coupling the panel to the block for closing the cavity.

8. The device of claim 7, further including the plurality of second connectors comprising two second connectors positioned singly proximate to the opposing termini of the cavity.

9. The device of claim 8, further including the second connector and the respective first connector comprising a cover hook and loop fastener.

10. The device of claim 1, further comprising:
a pair of first couplers, each first coupler being coupled to a respective opposing end of the block; and
a pair of second couplers, each second coupler being coupled to a respective opposing endpoint of the strap, the second couplers being complementary to the first couplers wherein each second coupler is positioned for selectively coupling to a respective first coupler for coupling the strap and the block around the waist of the user such that the block is positioned on the side of the torso of the user wherein the block is configured for abducting the shoulder of the user and wherein the cavity is configured for stowing the article.

11. The device of claim 10, further comprising:
each first coupler comprising a ring hingedly coupled to the respective opposing end of the block, the ring being squarely shaped; and
each second coupler comprising a strapping hook and loop fastener wherein a first element of the strapping hook and loop fastener is positioned adjacent to the respective opposing endpoint of the strap and a second element of the strapping hook and loop fastener is positioned on the strap distal from the first element such that the strap is positioned for inserting through the ring positioning the first element for selectively coupling to the second element for removably coupling the strap to the ring.

12. The device of claim 11, further including the second element being dimensionally longer than the first element such that the first element is selectively positionable on the second element for adjustably coupling the strap and the block around the waist of the user.

13. A shoulder abduction device comprising:
a block, the block being padded, the block comprising foamed elastomer such that the block is resiliently compressible, the block being substantially rectangularly box shaped, the block having a first face and a second face, the first face being convexly arcuate, the second face being concavely arcuate wherein the second face is configured abutting a side of a torso of a user;
a cavity extending into the block from an upper face of the block wherein the cavity is configured for stowing an article, the cavity having opposing termini, the opposing termini being arcuate;
a liner coupled to an interior surface of the block defined by the cavity, the liner comprising rubber wherein the liner is configured for dampening noise produced by movement of the article in the cavity and for protecting the article from damage;
a lid selectively couplable to the upper face of the block for closing the cavity, the lid comprising:
a panel, the panel being flexible,
a plurality of first connectors coupled to the panel, and
a plurality of second connectors coupled to the upper face of the block, the second connectors being complementary to the first connectors wherein each second connector is positioned for selectively coupling to a respective first connector for coupling the panel to the block for closing the cavity, the plurality of second connectors comprising two second connectors positioned singly proximate to the opposing termini of the cavity, the second connector and the respective first connector comprising a cover hook and loop fastener;
a strap having opposing endpoints, each opposing endpoint being selectively couplable to a respective opposing end of the block wherein the strap is configured for loopedly positioning around a waist of the user positioning the opposing endpoints for coupling to the block such that the block and the strap are coupled to the user and such that the block is positioned on the side of the torso of the user wherein the block is configured for abducting a shoulder of the user;
a pair of first couplers, each first coupler being coupled to a respective opposing end of the block, each first coupler comprising a ring hingedly coupled to the respective opposing end of the block, the ring being squarely shaped; and
a pair of second couplers, each second coupler being coupled to a respective opposing endpoint of the strap, the second couplers being complementary to the first couplers wherein each second coupler is positioned for selectively coupling to a respective first coupler for coupling the strap and the block around the waist of the user such that the block is positioned on the side of the torso of the user wherein the block is configured for abducting the shoulder of the user and wherein the cavity is configured for stowing the article, each second coupler comprising a strapping hook and loop fastener wherein a first element of the strapping hook and loop fastener is positioned adjacent to the respective opposing endpoint of the strap and a second element of the strapping hook and loop fastener is positioned on the strap distal from the first element such that the strap is positioned for inserting through the ring positioning the first element for selectively coupling to the second element for removably coupling the strap to the ring, the second element being dimensionally longer than the first element such that the first element is selectively positionable on the second element for adjustably coupling the strap and the block around the waist of the user.
